(19) United States
(12) Patent Application Publication
(10) Pub. No.: US 2022/0010059 A1
(43) Pub. Date: Jan. 13, 2022

(54) POLYHYDROXYALKANOATES EXTRACTION SYSTEM

(71) Applicant: National Chi Nan University, Nantou County (TW)

(72) Inventors: Yung-Pin TSAI, Nantou County (TW); Ku-Fan CHEN, Tainan (TW); Meng-Shan LU, Kaohsiung (TW); Chih-Chi YANG, Miaoli County (TW); Hao SHIU, New Taipei (TW)

(21) Appl. No.: 17/026,993

(22) Filed: Sep. 21, 2020

(30) Foreign Application Priority Data
Jul. 13, 2020 (TW) 109123564

(51) Int. Cl. C08G 63/89; C08G 63/06; C12P 7/62; C02F 11/06; C02F 11/20; C02F 3/12

(52) U.S. Cl. CPC C08G 63/89; C08G 63/06; C12P 7/625; C02F 3/1263; C02F 11/20; C02F 3/1215; C02F 11/06

(57) ABSTRACT

A polyhydroxyalkanoates extraction system comprises a pretreatment subsystem, an extraction subsystem and a recycling subsystem. The pretreatment subsystem comprises a fermentation device and an activation device so as to carry out a microorganism acclimation process. The extraction subsystem comprises a freezing device, a pretreatment device and an extraction device. The extraction subsystem is used for receiving a third sludge so that the third sludge is subjected to a freezing process, a pretreatment process, an extraction process and a purification process in the freezing device to form a polyhydroxyalkanoates mixture, and the extraction device performs a precipitation process to generate polyhydroxyalkanoates precipitate. The recycling subsystem comprises an aerobic sludge digestion device and a sequencing batch reactor activated sludge treatment device so as to carry out an aerobic sludge digestion process and a sequencing batch reactor activated sludge process.

### FIELD OF THE INVENTION

The present invention relates to an extraction system, and more particularly to a polyhydroxyalkanoates extraction system.

### BACKGROUND OF THE INVENTION

With the development of environmental issues, more and more attention has been paid to the development of biodegradable materials. Polyhydroxyalkanoates (PHAs) are important raw materials for biodegradable plastics, and have the properties of extensibility, thermoplasticity and so on, which are suitable for further processing and molding, and its properties are similar to those of polyethylene (PE) or polystyrene (PS).

Polyhydroxyalkanoates are common biopolymer products in cells of microorganism. Many microorganisms have limited growth elements such as nitrogen, phosphorus, sulfur, oxygen or magnesium, but in the presence of an external carbon source, they can absorb bases to synthesize polyhydroxyalkanoates. Polyhydroxyalkanoates are aliphatic polymers composed of carbon, hydrogen and oxygen, and are polymerized from hydroxyalkanoates (HAs) units. There are as many as 150 polyhydroxyalkanoates synthesized from different HAs.

However, existing polyhydroxyalkanoates production methods require more energy consumption and higher production costs. In addition, the extraction agent used in the existing process for extracting polyhydroxyalkanoates is high in price and has great harm to the environment. In view of the fact that high production cost is always the main factor hindering the popularization of biodegradable materials, reducing the production cost of polyhydroxyalkanoates and improving the extraction purity thereof so as to improve the economic benefits of industrial production of polyhydroxyalkanoates is actually the focus of attention of relevant persons in the art.

### SUMMARY OF THE INVENTION

The invention provides a polyhydroxyalkanoates (PHAs) extraction system which can effectively extract polyhydroxyalkanoates.

Other objects and advantages of the present invention can be further understood from the technical features disclosed herein.

The polyhydroxyalkanoates extraction system comprises a pretreatment subsystem, an extraction subsystem and a recycling subsystem. The pretreatment subsystem comprises a fermentation device and an activation device. The fermentation device includes a fermentation tank. A first sludge is placed in the fermentation device and fermented in the fermentation tank to form a fermentation broth. The activation device includes a thermostatic water tank and an oxygen supply device. A second sludge is placed in the activation device and diluted by water in the thermostatic water tank. The oxygen supply device supplies oxygen to the second sludge in the thermostatic water tank to activate the second sludge and form activated sludge. The activation device receives the fermentation broth and performs microorganism acclimation process with the activated sludge in the thermostatic water tank and to form a third sludge. The extraction subsystem comprises a freezing device, a pretreatment device and an extraction device. The extraction subsystem receives the third sludge and performs a freezing process with the third sludge in the freezing device to form a fourth sludge. The pretreatment device receives the fourth sludge and performs a pretreatment process to disrupt cells of microorganism in the fourth sludge. The extraction device comprises an extraction tank and a precipitation tank. The extraction device receives the fourth sludge and performs an extraction process and a purification process in the extraction tank to form a polyhydroxyalkanoates mixture and a first waste, wherein the extraction process is adding an aqueous solution of sodium hypochlorite (NaClO) into the fourth sludge to lyse the cell walls of microorganism and release polyhydroxyalkanoates. The purification process removes substances other than polyhydroxyalkanoates. The precipitation tank receives the polyhydroxyalkanoates mixture and performs a precipitation process to produce a polyhydroxyalkanoates precipitate and a second waste. The recycling subsystem comprises an aerobic sludge digestion device and a sequencing batch reactor activated sludge treatment device. The aerobic sludge digestion device receives part of the first waste and/or part of the second waste and performs a aerobic sludge digestion process. The sequencing batch reactor activated sludge treatment device receives part of the first waste and/or part of the second waste and performs a sequencing batch reactor activated sludge process.

In an embodiment of the invention, the extraction tank further comprises a stirrer and a liquid level controller.

In an embodiment of the invention, the pretreatment device comprises an ultrasonic pulverizer, the pretreatment process comprises an ultrasonic treatment process to treat the fourth sludge, and wherein an ultrasonic wave is applied to the fourth sludge by the ultrasonic pulverizer.

In an embodiment of the invention, the pretreatment process comprises placing the fourth sludge in an environment at a temperature above 30° C.

In an embodiment of the invention, the pretreatment process comprises adding the aqueous sodium hypochlorite solution into the fourth sludge.

In an embodiment of the invention, the pretreatment device comprises a high-voltage pulse generator, the pretreatment process comprises a high-voltage pulse extraction process performing on the fourth sludge, and the high-voltage pulse generator applies a high-voltage pulse electric field to the fourth sludge to disrupt the microorganism and release the polyhydroxyalkanoates.

In an embodiment of the invention, the extraction process comprises adding the aqueous solution of sodium hypochlorite into the fourth sludge to prepare a sludge mixture with a liquid-solid ratio of 0.67 mg/ml to 4 mg/ml, wherein the liquid-solid ratio is a ratio of a weight of a solid portion in the fourth sludge to a volume of the aqueous solution of sodium hypochlorite added.

In an embodiment of the invention, the extraction tank further comprises a centrifugation device, and the purification process comprises centrifuging the sludge mixture, washing with acetone, and centrifuging to remove materials other than polyhydroxyalkanoates.

In an embodiment of the invention, the first sludge is fermented in the fermentation tank at 40° C.-50° C. for 4-6 days to form the fermentation broth.

In an embodiment of the invention, the activation device comprises a dissolved oxygen monitor, and wherein if a saturated dissolved oxygen (DO) of the activated sludge reaches 75%-85% (6.18 mg/L-7.01 mg/L), the fermentation broth is added to synthesize polyhydroxyalkanoates, and the fermentation broth is added again if the saturated dissolved oxygen of the activated sludge drops to 65%-75% (5.36 mg/L-6.18 mg/L), then repeating 5-10 times to form the third sludge.

In an embodiment of the invention, after the pretreatment process, a surfactant solution is added to the fourth sludge, and the surfactant solution is an aqueous solution of sodium dodecyl sulfate (SDS) or sodium dodecyl sulfonate (SDS′).

On the basis of the previous disclosure, in the polyhydroxyalkanoates extraction system of the present invention, the pretreatment subsystem, the extraction subsystem and the recycling subsystem are arranged, so that the extraction efficiency of polyhydroxyalkanoates can be greatly improved, the extraction cost is greatly reduced, the polyhydroxyalkanoates with high purity can be effectively extracted, and the polyhydroxyalkanoates can be extracted from waste sludge with good economic benefits.

In order to make the above-mentioned features and advantages of the present invention more clear and understandable, the following specific examples are given in conjunction with the accompanying drawings to describe in detail as follows.

### BRIEF DESCRIPTION OF THE DRAWINGS

FIG. 1 is a schematic diagram of a polyhydroxyalkanoates extraction system according to one embodiment of the present invention.

FIG. 2 is a schematic diagram of a polyhydroxyalkanoates extraction system according to another embodiment of the present invention.

FIG. 3 is a schematic diagram of a polyhydroxyalkanoates extraction system according to another embodiment of the present invention.

### DETAILED DESCRIPTION OF THE PREFERRED EMBODIMENTS

The foregoing and other aspects, features and advantages of the invention will be clear from the following detailed description of preferred embodiments, taken in conjunction with the accompanying drawings. Directional terms mentioned in the following examples, for example: up, down, left, right, front or rear, etc., are directions only with reference to the appended drawings. Accordingly, directional terminology is used for the purpose of description and not of limitation.

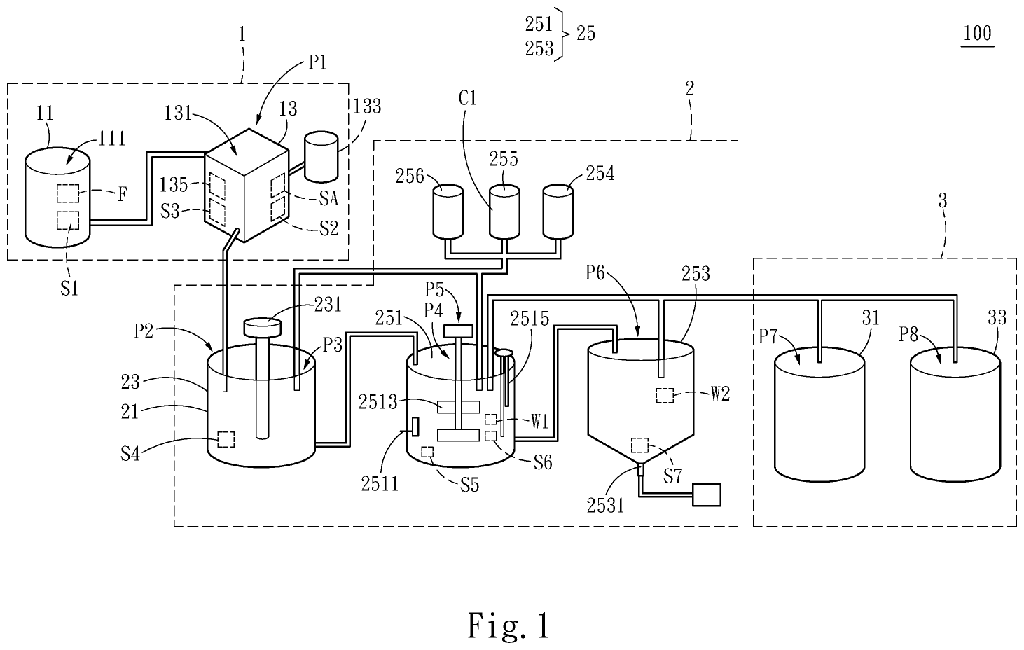

Referring to FIG. 1, a schematic diagram of a polyhydroxyalkanoates extraction system 100 according to one embodiment of the present invention is shown. The polyhydroxyalkanoates extraction system 100 includes a pretreatment subsystem 1, an extraction subsystem 2, and a recycling subsystem 3. The pretreatment subsystem 1 cultures polyhydroxyalkanoates (PHAs) with the sludge. The extraction subsystem 2 extracts polyhydroxyalkanoates from the sludge. The recycling subsystem 3 recycles the waste generated in the treatment process. Therefore, the extraction efficiency of polyhydroxyalkanoates is greatly improved. Specific operational details are described in detail below.

The pretreatment subsystem 1 comprises a fermentation device 11 and an activation device 13. The fermentation device 11 includes a fermentation tank 111. A first sludge S1 is placed in the fermentation device 11 and fermented in the fermentation tank 111 to form a fermentation broth F.

The activation device 13 includes a thermostatic water tank 131 and an oxygen supply device 133. A second sludge S2 is placed in the activation device 13 and the second sludge S2 diluted by the water in the thermostatic water tank 131. The oxygen supply device 133 supplies oxygen to the second sludge S2 in the thermostatic water tank 131 to activate the second sludge S2 and to form an activated sludge SA. The activation device 13 receives the fermentation broth F and performs a microorganism acclimation process P1 with the activated sludge SA in the thermostatic water tank 131 and then to form a third sludge S3.

The extraction subsystem 2 comprises a freezing device 21, a pretreatment device 23 and an extraction device 25. The extraction subsystem 2 receives the third sludge S3 and performs a freezing process P2 with the third sludge S3 in the freezing device 21 to form a fourth sludge S4. The freezing process P2 freezes the third sludge S3 to stop metabolism of microorganism in the third sludge S3. The pretreatment device 23 receives the fourth sludge S4 which is frozen and performs a pretreatment process P3 to disrupt cells of microorganism (not shown) in the fourth sludge S4. In the embodiment shown in FIG. 1, the freezing device 21 is provided on the pretreatment device 23 as an example, but it is not limited thereto. In other embodiments, the freezing device 21 and the pretreatment device 23 are two separate devices. The freezing device 21 may be implemented with any possible cooler.

The extraction device 25 includes an extraction tank 251 and a precipitation tank 253. The extraction device 25 receives the fourth sludge S4 and performs an extraction process P4 and a purification process P5 in the extraction tank 251 to form a polyhydroxyalkanoates mixture S6 and a first waste W1. The extraction process P4 comprises adding an aqueous solution C1 of sodium hypochlorite (NaClO) into the fourth sludge S4 to lyse cell walls of microorganism in the fourth sludge S4 and release polyhydroxyalkanoates. Next, the purification process P5 is performed in the extraction tank 251. The purification process P5 removes substances other than the polyhydroxyalkanoates.

In this embodiment, the precipitation tank 253 receives the polyhydroxyalkanoates mixture S6 and performs a precipitation process P6 to precipitate and separate the polyhydroxyalkanoates mixture S6 and to produce a polyhydroxyalkanoates precipitate S7 and a second waste W2. For example, the polyhydroxyalkanoates precipitate S7 is collected from a collection port 2531 of the precipitation tank 253, but it is not limited thereto. Therefore, the extraction subsystem 2 effectively extracts the polyhydroxyalkanoates precipitate S7 with high purity.

In this embodiment, the recycling subsystem 3 includes an aerobic sludge digestion device 31 and a sequencing batch reactor (SBR) activated sludge treatment device 33. The aerobic sludge digestion device 31 receives part of the first waste W1 and/or part of the second waste W2 to perform an aerobic sludge digestion process P7 on part of the first waste W1 and/or part of the second waste W2. The aerobic sludge digestion process P7 is a process for stabilizing organic sludge (i.e., part of the first waste W1 and/or part of the second waste W2) by microorganisms in an aerobic state. When sludge is aerated, organic substances in the sludge are oxidized into carbon dioxide, water and ammonia via aerobic microorganisms. Then, ammonia is further oxidized to nitrates. Namely, the aerobic sludge digestion process P7 reduces the amount of sludge, improve the dehydration property of the sludge, stabilize the sludge, avoid decomposing and deodorizing in a subsequent treatment process, and effectively treat wastes.

The sequencing batch reactor activated sludge treatment device 33 receives part of the first waste W1 and/or part of the second waste W2 to perform a sequencing batch reactor activated sludge process P8. The sequencing batch reactor activated sludge process P8 is an activated sludge wastewater treatment technology that operates in a batch aeration mode, and thus part of the first waste W1 and/or part of the second waste W2 can be treated effectively. Accordingly, the polyhydroxyalkanoates extraction system 100 of the present embodiment not only can greatly improve the extraction efficiency of polyhydroxyalkanoates, but also can effectively treat the wastes.

In addition, the first sludge S1 and/or the second sludge S2 can be taken from any type of sludge, such as domestic sludge, hospital sludge, fermentation industry sludge, animal husbandry sludge and the like.

In one embodiment, the first sludge S1 is fermented in the fermentation tank 111 to form the fermentation broth F with a fermentation reaction to directly obtain a carbon source for feeding microorganisms from the first sludge S1. In one embodiment, the first sludge S1 is fermented at 40° C.-50° C. for 4-6 days. In another embodiment, the first sludge S1 is fermented at 40° C. for 5 days.

In one embodiment, before the second sludge S2 is placed in the thermostatic water tank 131 to be diluted by water, the second sludge S2 is screened (e.g., to pass through a sieve) to remove large impurities, such as stones and leaves. Then, pH value of the second sludge S2 is adjusted to 10.5-11.5, preferably the pH value is 11, and the second sludge S2 is left to rest at 4° C. for at least 12 hours. Therefore, the follow-up acclimation of polyhydroxyalkanoates has the best effect.

In addition, in the process of forming the activated sludge SA, the purpose is to sufficiently aerate the second sludge S2 with oxygen to activate the second sludge S2, thereby providing oxygen to an aerobic microorganism population (not shown) in the second sludge S2 for a subsequent acclimation treatment. The second sludge S2 is placed in the thermostatic water tank 131 to be diluted by water, so that a better aeration activation effect can be achieved. In one embodiment, reverse osmosis (RO) water is mixed with the second sludge S2 to dilute the second sludge S2 in a ratio of 1:1. Thus, after the diluted second sludge S2 is aerated with oxygen, oxygen can be uniformly distributed in the second sludge S2 so as to achieve a better activation effect. In one embodiment, the oxygen supply device 133 is implemented with any possible oxygen supply, and it is not limited thereto.

In one embodiment, the activation device 13 further comprises a dissolved oxygen monitor 135 for monitoring dissolved oxygen in the thermostatic water tank 131. In the microorganism acclimation process P1 that the activation device 13 receives the fermentation broth F, if the saturated dissolved oxygen (DO) of the activated sludge SA reaches 75%-85% (6.18 mg/L-7.01 mg/L), the fermentation broth F is added to synthesize polyhydroxyalkanoates; and the fermentation broth F is added again if the saturated dissolved oxygen of the activated sludge SA drops to 65%-75% (5.36 mg/L-6.18 mg/L), then repeating the aforementioned steps 5-10 times to form the third sludge S3. Thereby, the fermentation broth F is added into the activated sludge SA for microbial acclimation to promote cell synthesis and accumulation of polyhydroxyalkanoates.

Through the arrangement of the dissolved oxygen monitor 135, the microbial acclimation method monitors the condition that the microorganisms consume the external carbon source through a simple process, so that the external carbon source (namely the fermentation broth F) can be accurately added again, and therefore the food to microorganism ratio (F/M) can be controlled to be 0.19±0.08. Thus, the high-performance feast and famine cycling culture can be achieved. Therefore, the synthesis and storage rate of polyhydroxyalkanoates in cells are improved. The efficiency of culturing polyhydroxyalkanoates by the pretreatment subsystem 1 can be greatly improved.

In addition, any possible piping and flow control mechanisms may be provided between the fermentation device 11 and the activation device 13, such that the fermentation broth F can be automatically added into the activated sludge SA on demand.

In addition, when the fermentation broth F is added to the activated sludge SA to synthesize polyhydroxyalkanoates, the fermentation broth F is added at a temperature ranging from 23° C. to 26° C. and a pH value ranging from 8.5 to 9.5, but it is not limited thereto.

In the present embodiment, the pretreatment device 23 includes an ultrasonic pulverizer 231. The pretreatment process P3 comprises an ultrasonic treatment process to treat the fourth sludge S4, wherein an ultrasonic wave is applied to the fourth sludge S4 by the ultrasonic pulverizer 231 so as to crush the fourth sludge S4 and disrupt cells of microorganism in the fourth sludge S4, thereby facilitating subsequent extraction operation.

In another embodiment, the pretreatment process P3 places the fourth sludge S4 in an environment at a temperature above 30° C. to disrupt cells of microorganism within the fourth sludge S4 for subsequent extraction operations.

In another embodiment, the pretreatment process P3 includes adding the aqueous solution C1 of sodium hypochlorite (NaClO) into the fourth sludge S4 to disrupt cells of microorganism in the fourth sludge S4 for subsequent extraction operations.

In one embodiment, after the pretreatment process P3, for example, a surfactant solution is also added to the fourth sludge S4. The surfactant solution is, for example, an aqueous solution of sodium dodecyl sulfate (SDS) or sodium dodecyl sulfonate (SDS′). Accordingly, the surfactant molecules will enter the phospholipid bilayer of the cell membrane of the microbial cell, and as the concentration of the surfactant solution increases, more and more surfactant molecules will be bound to the phospholipid bilayer, thereby increasing the volume of the cell membrane. When the surfactant molecules bound to the phospholipid bilayer have reached saturation, continued increase of the surfactant molecules will form a large number of micelles with the phospholipid bilayer and cause cell membrane disruption. Thus, the polyhydroxyalkanoates in the cell are released. In addition, considering that the surfactant solution would cause protein denaturation, solubilization and the like, even if the amount of the surfactant solution does not reach saturation, the structure of the cell membrane can be influenced to be disrupted easily, and the release of the PHAs is also facilitated.

In one embodiment, the aqueous solution of sodium dodecyl sulfate includes a concentration of 1-10 w/v %. In one embodiment, the surfactant solution added into the fourth sludge S4 is carried out at a temperature of 30° C.-40° C.

In one embodiment, the extraction tank 251 further includes a stirrer 2513 and a liquid level controller 2515. The stirrer 2513 mixes solutions in the extraction tank 251. The liquid level controller 2515 controls the level of solution within the extraction tank 251. Thereby, the efficiency and accuracy of extraction in the extraction tank 251 can be improved.

In one embodiment, the extraction process P4 includes adding the aqueous solution C1 of sodium hypochlorite (NaClO) into the fourth sludge S4 to prepare a sludge mixture S5 with a liquid-solid ratio of 0.67 mg/ml to 4 mg/ml, such that cell walls of microorganism in the fourth sludge S4 are lysed and polyhydroxyalkanoates are released. In detail, the liquid-solid ratio is a ratio of a weight of a solid portion in the fourth sludge S4 to a volume of the added aqueous solution C1 of sodium hypochlorite.

In an embodiment, the liquid-solid ratio of the sludge mixture S5 is 0.67 mg/ml-0.95 mg/ml. In one embodiment, the concentration of the aqueous solution C1 of sodium hypochlorite is 10 v/v %-60 v/v %. In one embodiment, the concentration of the aqueous solution C1 of sodium hypochlorite is 10 v/v %-100 v/v %. In one embodiment, the extraction process P4 is performed at a temperature of 37° C.

In addition, the extraction subsystem 2 also includes a plurality of tanks 254, 255, 256. In one embodiment, the tank 254 stores water, the tank 255 stores the aqueous solution C1 of sodium hypochlorite (NaClO), and the tank 256 store the aqueous solution of sodium dodecyl sulfate (SDS) or sodium dodecyl sulfonate (SDS′). Thus, the plurality of tanks 254, 255, 256 provide the raw materials required by each process.

In one embodiment, the extraction tank 251 further includes a centrifugation device 2511. The purification process P5 is performed by centrifuging the sludge mixture S5 and removing the supernatant (i.e., the first waste W1), followed by washing with acetone and centrifuging to remove materials other than the polyhydroxyalkanoates. The centrifugation device 2511 may be implemented with any possible type of centrifuge and it is not so limited. In addition, in the precipitation process P6, the precipitation tank 253 also includes a centrifugation device (not shown) for centrifuging the polyhydroxyalkanoates mixture S6 to produce the polyhydroxyalkanoates precipitate S7 and the second waste W2.

In another embodiment, prior to performing the purification process P5, the sludge mixture S5 is centrifuged and removed the supernatant (i.e., the first waste W1), followed by adding ice-ethanol to isolate the polyhydroxyalkanoates to remove materials other than the polyhydroxyalkanoates.

In this embodiment, the flow/movement of the substances between the devices may be accomplished through any possible line, valve body, and/or automatic/manual fluid controller, which are not described in detail.

By limiting the range of liquid-solid ratios of the sludge mixture S5 with the assistance of the freezing process P2, the pretreatment process P3, and the purification process P5, high purity polyhydroxyalkanoates can be extracted directly from the waste sludge (i.e., the first sludge S1 and/or the second sludge S2) without pure culture. Therefore, the reuse value of the waste sludge can be increased, environmental protection is facilitated, and the cost of raw materials to be extracted can be reduced. In addition, using the aqueous solution C1 of sodium hypochlorite (NaClO) as the main extractant is relatively friendly to the environment and thus reducing the harm to the environment. Therefore, the polyhydroxyalkanoates extraction system 100 has the advantages of being environment-friendly, low in extraction cost, capable of smoothly obtaining high-purity polyhydroxyalkanoates raw materials, and the like. Therefore, the application popularization of the polyhydroxyalkanoates is facilitated, and the polyhydroxyalkanoates can be further developed into a system for producing the polyhydroxyalkanoates industrially and has deep practical value.

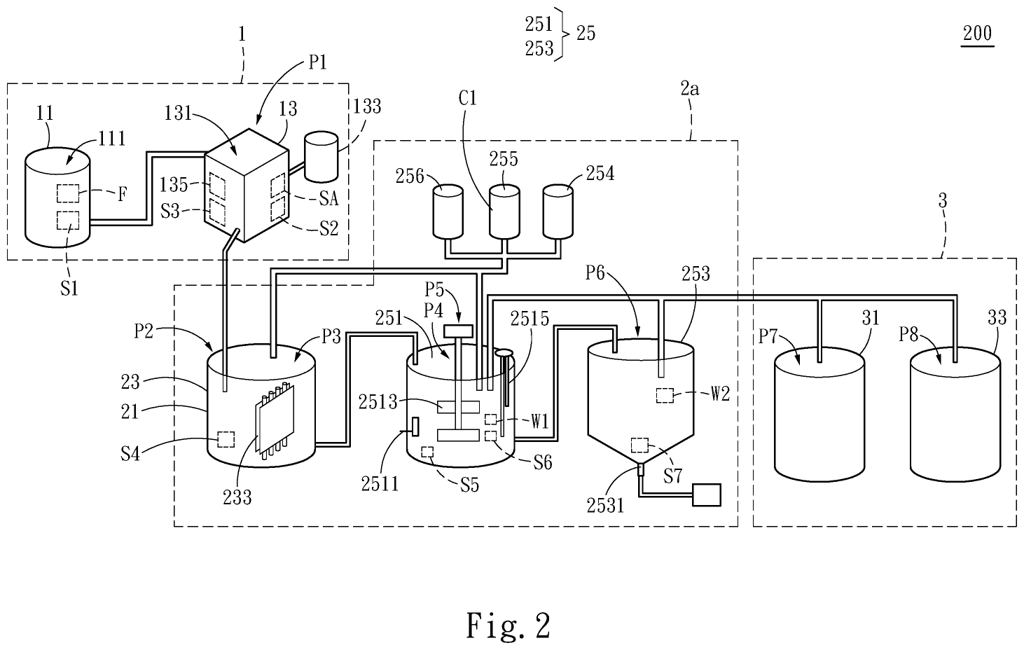

Referring to FIG. 2, a schematic diagram of a polyhydroxyalkanoates extraction system 200 in accordance with another embodiment of the present invention is shown. The polyhydroxyalkanoates extraction system 200 of this embodiment is similar in structure and function to the polyhydroxyalkanoates extraction system 100 shown in FIG. 1. This embodiment differs from the embodiment shown in FIG. 1 in that: a pretreatment device 23 of an extraction subsystem 2a comprises a high-voltage pulse generator 233.

The pretreatment process P3 includes a high-voltage pulse extraction process performing on the fourth sludge S4. The high-voltage pulse generator 233 applies a high-voltage pulse electric field (not shown) to the fourth sludge S4 to disrupt microorganisms in the fourth sludge S4 and release polyhydroxyalkanoates.

In one embodiment, the high-voltage pulse electric field is ranged between 50V and 400V, the application time of the high-voltage pulse electric field is ranged between 5 seconds and 90 seconds, and the application frequency of the high-voltage pulse electric field is between 500 Hz and 1000 Hz.

In another embodiment, the high-voltage pulse generator applies the high-voltage pulse electric field to the fourth sludge S4 at 100V to 400V with a frequency of 500 Hz to 1000 Hz for 15 seconds to 60 seconds.

In the present embodiment, a large pulse electric field for a very short time is used to make the cells of microorganisms temporarily produce some micro pores by high-voltage electroporation through the high-voltage pulse generator 233, thereby achieving the effect of polyhydroxyalkanoates extraction.

In one embodiment, the pretreatment device 23 includes both the high-voltage pulse generator 233 and the ultrasonic pulverizer 231, thereby achieving the effect of polyhydroxyalkanoates extraction.

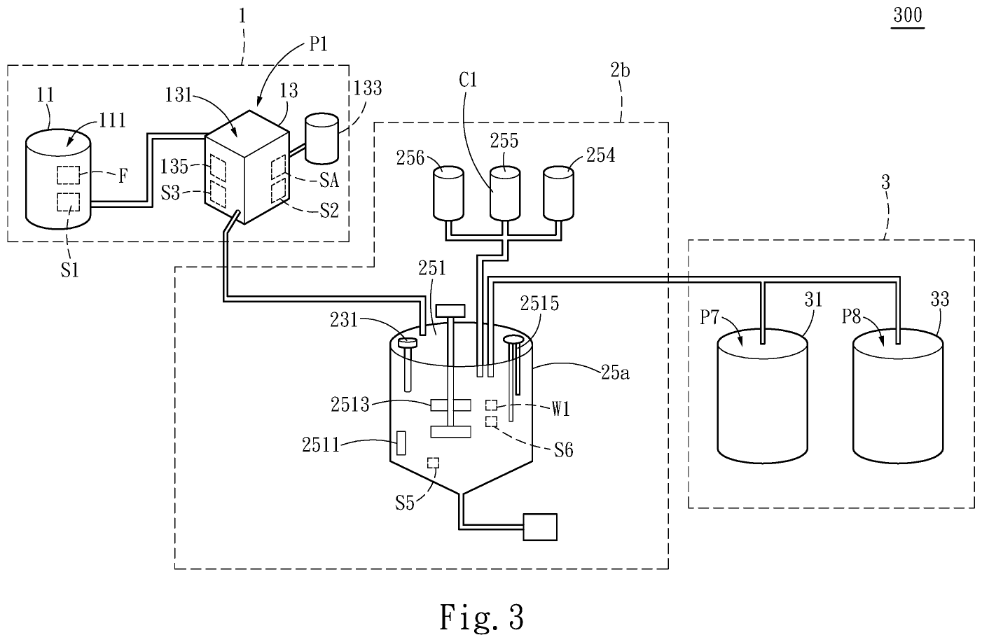

Referring to FIG. 3, a schematic diagram of a polyhydroxyalkanoates extraction system 300 in accordance with another embodiment of the present invention is shown. The polyhydroxyalkanoates extraction system 300 of this embodiment is similar in structure and function to the polyhydroxyalkanoates extraction system 100 shown in FIG. 1. This embodiment differs from the embodiment shown in FIG. 1 in that: in the polyhydroxyalkanoates extraction system 300, an extraction subsystem 2b integrates the extraction tank 251 and the precipitation tank 253 shown in FIG. 1 as an extraction device 25a, and integrates the freezing device 21 and the pretreatment device 23 shown in FIG. 1 into the extraction device 25a. That is, the extraction device 25a simultaneously functions as the freezing device 21, the pretreatment device 23, the extraction tank 251 and the precipitation tank 253 of the embodiment shown in FIG. 1. Therefore, the volume and complexity of the polyhydroxyalkanoates extraction system 300 are greatly reduced, and the cost is reduced.

In summary, the polyhydroxyalkanoates extraction system provided by the embodiments of the invention greatly improve the extraction efficiency of polyhydroxyalkanoates, greatly reduce the extraction cost, effectively extract high-purity polyhydroxyalkanoates by arranging the pretreatment subsystem, the extraction subsystem and the recycling subsystem, and has better economic benefit in extracting polyhydroxyalkanoates from waste sludge.

What is claimed is:

1. A polyhydroxyalkanoates (PHAs) extraction system comprising:
a pretreatment subsystem including:
a fermentation device, including a fermentation tank, a first sludge placed in the fermentation device and fermented in the fermentation tank to form a fermentation broth; and
an activation device, including a thermostatic water tank and an oxygen supply device, a second sludge placed in the activation device and diluted by water in the thermostatic water tank, the oxygen supply device supplying oxygen to the second sludge in the thermostatic water tank to activate the second sludge and form an activated sludge, and the activation device receiving the fermentation broth and performing a microorganism acclimation process with the activated sludge in the thermostatic water tank and to form a third sludge;
an extraction subsystem including:
a freezing device, receiving the third sludge and performing a freezing process with the third sludge in the freezing device to form a fourth sludge;
a pretreatment device, receiving the fourth sludge and performing a pretreatment process to disrupt cells of microorganism in the fourth sludge; and
an extraction device, including an extraction tank and a precipitation tank, the extraction device receiving the fourth sludge and performing an extraction process and a purification process in the extraction tank to form a polyhydroxyalkanoates mixture and a first waste, wherein the extraction process is adding an aqueous solution of sodium hypochlorite (NaClO) into the fourth sludge to lyse cell walls of microorganism and release polyhydroxyalkanoates, and wherein the purification process is removing substances other than polyhydroxyalkanoates, the precipitation tank receiving the polyhydroxyalkanoates mixture and performing a precipitation process to produce a polyhydroxyalkanoates precipitate and a second waste; and
a recycling subsystem including:
an aerobic sludge digestion device, receiving part of the first waste and/or part of the second waste and performing an aerobic sludge digestion process; and
a sequencing batch reactor activated sludge treatment device, receiving part of the first waste and/or part of the second waste and performing a sequencing batch reactor activated sludge process.

2. The polyhydroxyalkanoates extraction system according to claim 1, wherein the extraction tank further comprises a stirrer and a liquid level controller.

3. The polyhydroxyalkanoates extraction system according to claim 1, wherein the pretreatment device comprises an ultrasonic pulverizer, the pretreatment process comprises an ultrasonic treatment process to treat the fourth sludge, and wherein an ultrasonic wave is applied to the fourth sludge by the ultrasonic pulverizer.

4. The polyhydroxyalkanoates extraction system according to claim 1, wherein the pretreatment process comprises placing the fourth sludge in an environment at a temperature above 30° C.

5. The polyhydroxyalkanoates extraction system according to claim 1, wherein the pretreatment process comprises adding the aqueous solution of sodium hypochlorite into the fourth sludge.

6. The polyhydroxyalkanoates extraction system according to claim 1, wherein the pretreatment device comprises a high-voltage pulse generator, the pretreatment process comprises a high-voltage pulse extraction process performing on the fourth sludge, and the high-voltage pulse generator applies a high-voltage pulse electric field to the fourth sludge to disrupt microorganisms and release polyhydroxyalkanoates.

7. The polyhydroxyalkanoates extraction system according to claim 1, wherein the extraction process comprises adding the aqueous solution of sodium hypochlorite into the fourth sludge to prepare a sludge mixture with a liquid-solid ratio of 0.67 mg/ml 4 mg/ml, wherein the liquid-solid ratio is a ratio of a weight of a solid portion in the fourth sludge to a volume of the aqueous solution of sodium hypochlorite added.

8. The polyhydroxyalkanoates extraction system according to claim 7, wherein the extraction tank further comprises a centrifugation device, and the purification process comprises centrifuging the sludge mixture, washing with acetone, and centrifuging to remove materials other than polyhydroxyalkanoates.

9. The polyhydroxyalkanoates extraction system according to claim 1, wherein the first sludge is fermented in the fermentation tank at 40° C.-50° C. for 4-6 days to form the fermentation broth.

10. The polyhydroxyalkanoates extraction system according to claim 1, wherein the activation device comprises a dissolved oxygen monitor, and wherein if a saturated dissolved oxygen (DO) of the activated sludge reaches 75%-85% (6.18 mg/L-7.01 mg/L), the fermentation broth is added to synthesize polyhydroxyalkanoates, and the fermentation broth is added again if the saturated dissolved oxygen of the activated sludge drops to 65%-75% (5.36 mg/L-6.18 mg/L), then repeating 5-10 times to form the third sludge.

11. The polyhydroxyalkanoates extraction system according to claim 1, wherein after the pretreatment process, a surfactant solution is added to the fourth sludge, and the surfactant solution is an aqueous solution of sodium dodecyl sulfate (SDS) or sodium dodecyl sulfonate (SDS′).
